(19) United States
(12) Patent Application Publication
(10) Pub. No.: US 2022/0002551 A1
(43) Pub. Date: Jan. 6, 2022

(54) A NOVEL CLASS OF PIGMENTS IN ASPERGILLUS

(71) Applicant: DANMARKS TEKNISKE UNIVERSITET, Kgs. Lyngby (DK)

(72) Inventors: Thomas Isbrandt Petersen, Virum (DK); Phillip Kroll-Møller, Copenhagen NV (DK); Thomas Ostenfeld Larsen, Holte (DK); Anders Sebastian Rosenkrans Ødum, Nærum (DK)

(21) Appl. No.: 17/291,272

(22) PCT Filed: Nov. 8, 2019
(86) PCT No.: PCT/EP2019/080647

§ 371 Date: May 4, 2021

(51) Int. Cl. C09B 61/00; C12P 17/18

(52) U.S. Cl. CPC C09B 61/00; C12R 2001/66; C12P 17/181

(57) ABSTRACT

The invention provides a novel class of natural red azaphilone pigments: cavernamines and their hydroxyl-derivatives; as well as the orange/yellow precursor cavernine. Additionally, methods for their production by fermentation using a fungal strain belonging to the species Aspergillus cavernicola, is provided; and further the use of the novel pigments as a colouring agent for food items and/or non-food items, and for cosmetics. The cavernamine pigments have the structure of Formula I or II, the hydroxyl-derivative of said cavernamine pigment has the structure of Formula III: Cavernine pigments having the structure of Formula IV or V are precursors f the cavernamine pigments I-III above.

### FIELD OF THE INVENTION

The invention provides a novel class of natural red azaphilone pigments: cavernamines and their hydroxyl-derivatives; as well as their respective orange/yellow precursor cavernine. Additionally, methods for their production by fermentation using Aspergillus cavernicola, is provided; and further the use of the novel pigments, and a kit comprising the same, as a colouring agent for food items and/or non-food items, and for cosmetics.

### BACKGROUND OF THE INVENTION

Natural food colorants are increasingly sought after due to growing consumer awareness of potential harmful effects of synthetic colorants^{1,2}. In view of the increasing recognition of a link between diet and health, the food additive industry faces new challenges in providing natural color alternatives. So far most industrially used natural colorants are extracted directly from natural sources e.g. betanin (beet root Beta vulgaris extract), lycopene (tomato Solanum lycopersicum extract) or carminic acid (extracted from the female insect Dactylopius coccus³). Their production is highly dependent on the supply of raw ingredients, which are subject to seasonal variation both in regards to quantity and quality⁴. These limitations can be overcome by exploring new sources for natural pigments such as microorganisms⁵. Fungi are known to naturally biosynthesize and excrete diverse classes of secondary metabolites including pigments within a broad range of colors⁶.

Monascus is a pigment-producing fungal genus that has long been used for the manufacture of traditional foods in Asian countries⁷. Pigments from Monascus are referred to as “Monascus pigments”, which are a mixture of azaphilones including yellow, orange, and red constituents.

The use of species of Monascus for the production of Monascus pigments results in a cocktail of different Monascus pigments⁸, having a range of hues, whose composition is difficult to control and can vary from batch-to-batch. In addition, species of Monascus are known to produce mycotoxins, such as citrinin⁹, which causes diverse toxic effects, including nephrotoxic, hepatotoxic and cytotoxic effects and which excludes their use for industrial purposes in western countries. From an industrial perspective it would be highly preferable to produce these component pigments individually by fermentation, where the individual species of pigment produced was free of mycotoxins, such that the pigment can easily be extracted and recovered without the need for multiple and possibly complex purification steps. Among the important uses of natural pigments are as food additives; where water soluble pigments are highly desirable.

### SUMMARY OF THE INVENTION

According to a first aspect, the present invention provides a cavernamine pigment having the structure of Formula I or II:

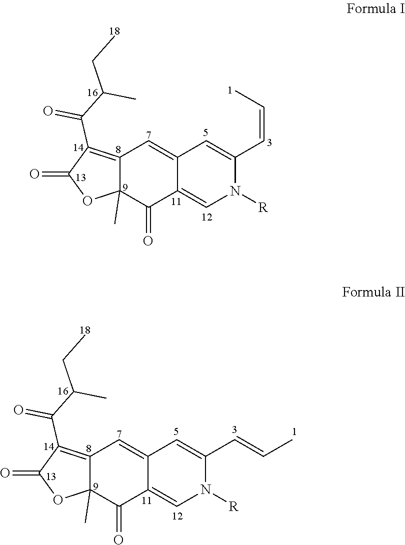

wherein R is hydrogen, or N—R is selected from among, an amino acid, a peptide, an amino sugar and a primary amine.

Preferably, N—R of Formula I is an amino acid selected from the group consisting of: L-alanine, L-arginine, L-asparagine, L-aspartate, L-cysteine, L-glutamine, L-glutamate, L-glycine, L-histidine, L-isoleucine, L-leucine, L-lysine, L-methionine, L-phenylalanine, L-proline, L-serine, L-threonine, L-tryptophan, L-tyrosine, L-valine and L-ornithine.

According to a second aspect, the invention provides a hydroxyl-cavernamine having the structure of formula III:

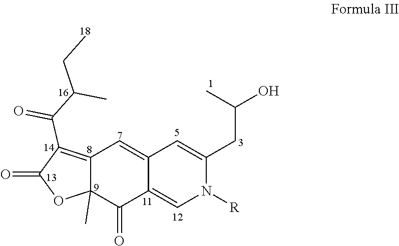

wherein R is hydrogen, or N—R is selected from among, an amino acid, a peptide, an amino sugar and a primary amine; and wherein said hydroxy-cavernamine is a hydroxyl-derivative of the cavernamine of the first aspect of the invention.

Preferably, N—R of Formula III is an amino acid selected from the group consisting of: L-alanine, L-arginine, L-asparagine, L-aspartate, L-cysteine, L-glutamine, L-glutamate, L-glycine, L-histidine, L-isoleucine, L-leucine, L-lysine, L-methionine, L-phenylalanine, L-proline, L-serine, L-threonine, L-tryptophan, L-tyrosine, L-valine and L-ornithine.

According to a third aspect, the invention provides a cavernine pigment having the structure of Formula IV or Formula V:

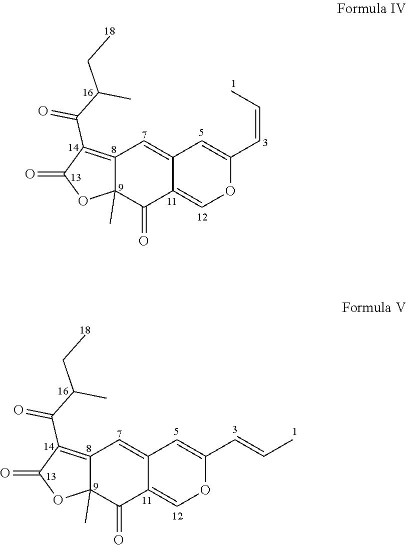 [SMILES: C/C=C\C1=CC2=CC3=C(C(=O)C(C)CC)C(=O)OC3(C)C(=O)C2=CO1 ; C/C=C/C1=CC2=CC3=C(C(=O)C(C)CC)C(=O)OC3(C)C(=O)C2=CO1]

wherein said cavernine pigment is a precursor of the cavernamine pigment of the first aspect of the invention and/or the hydroxyl-cavernaine of the second aspect of the invention.

According to a fourth aspect, the invention provides a method for producing a cavernamine pigment and/or a hydroxyl-derivative of said cavernamine pigment by fermentation, comprising the steps of:
a. providing spores or mycelia of a strain Aspergillus cavernicola,
b. cultivating said spores or mycelia in a liquid growth medium comprising a nitrogen source,
c. recovering the cavernamine pigment and/or its hydroxyl-derivative produced during said cultivating in step (b), and
d. optionally isolating said cavernamine pigment and/or its hydroxyl-derivative

Preferably, the sole nitrogen source in step (b) is a compound selected from the group consisting of a single amino acid, a peptide, an amino sugar and a primary amine.

The invention further provides a method for producing a cavernamine pigment and/or a hydroxyl-derivative of said cavernamine by fermentation comprising the additional step of:
a′) cultivating the spores or mycelia of step (a) in a preliminary liquid growth medium, wherein the sole nitrogen source of said preliminary liquid growth medium is an inorganic nitrogen source; and
wherein said step (a′) is followed by step (b).

The invention further concerns the use of a cavernamine pigment of Formula I or II, a hydroxyl-cavernamine of Formula III, and/or a cavernine of Formula IV or V as a colouring agent for any one of a food, a non-food product and a cosmetic;

Additionally, the invention concerns a kit of parts for coloring a composition, wherein the kit comprises (i) at least one cavernamine pigment of Formula I or II, at least one hydroxyl-cavernamine of Formula III and/or at least one cavernine of Formula IV or V, and (ii) a stabilizing agent, wherein the pigment is supplied in a container, wherein the composition is selected from among a food, a non-food product and a cosmetic.

### DESCRIPTION OF THE INVENTION

### Figures

FIG. 1: Structure of (A) cavernamine pigment (Formula I and II), (B) hydroxy-derivative of carvermine (Formula III), and (C) cavernine pigment (Formula IV and V).

FIG. 2: Diagram showing Base Peak Chromatogram (BPC) and UV-Chromatogram (EWC, measured at 520 nm) of compounds extracted from initial screening of A. cavernicola grown on Czapek Dox yeast extract agar (CYA) plates or in one-step liquid fermentation broth (as defined in example 1.7). (A) A. cavernicola IBT32660: 1) BPC of CYA plate extract. 2) EWC (520 nm) of CYA plate extract. 3) BPC of Czapek Dox broth extract, and 4) EWC (520 nm) of Czapek Dox broth extract. (B) A. cavernicola IBT23158: 1) BPC of CYA plate extract, 2) EWC (520 nm) of CYA plate extract, 3) BPC of Czapek Dox broth extract, and 4) EWC (520 nm) of Czapek Dox broth extract. The vertical dashed line in (A) and (B) indicates the yellow/orange precursor cavernine.

FIG. 3: Diagram showing EWC chromatograms of compounds extracted from cultivation medium derived from (A) A. carvernicola strain IBT32660, or (B) A. cavernicola strain IBT23158 grown on Czapek Dox media supplemented with amino acids leucine, histidine, valine, arginine, or tryptophan. Asterisk* indicates the expected cavernamine amino acid derivatives; cross^{†} indicates hydroxy-derivatives of the cavernamines; the vertical dashed line indicates the yellow/orange precursor cavernine; all verified by MS.

FIG. 4: Graphical presentation of the absorbance spectra of (A) cavernine and (B) cis-cavernamine-L.

FIG. 5: Pigment production (absorbance 520 nm, dark grey columns) and biomass formation (g/l, light grey columns) by A. cavernicola IBT32660 cultured at different pH.

FIG. 6: (A) Diagram showing ¹H and ¹³C NMR shifts for trans-cavernamine; asterisk indicates no signal detected. (B) Diagram showing the chemical structure of trans-cavernamine.

FIG. 7: (A) Diagram showing ¹H and ¹³C NMR shifts for cis-cavernamine; asterisk indicates no signal detected; (B) Diagram showing the chemical structure of cis-cavernamine.

FIG. 8: (A) Diagram showing ¹H and ¹³C NMR shifts for cis-cavernamine-L; asterisk indicates no signal detected. (B) Diagram showing the chemical structure of cis-cavernamine-L.

FIG. 9: (A) Diagram showing ¹H and ¹³C NMR shifts for trans-cavernine; asterisk indicates no signal detected. (B) Diagram showing the chemical structure of trans-carvernine.

FIG. 10: (A) Diagram showing ¹H and ¹³C NMR shifts for hydroxy-cavernamine-H; asterisk indicates no signal detected. (B) Diagram showing the chemical structure of hydroxy-cavernamine-H.

FIG. 11: From left to right: Skim milk 0.1% as control, skim milk 0.1% with 28 ppm of cavernamine-L, skim milk 0.1% with 140 ppm of cavernamine-L, and skim milk 0.1% with 280 ppm of cavernamine-L.

FIG. 12: Left: Skyr control, Right: Skyr with 46 ppm of cavernamine-L.

FIG. 13: From left to right: Epoxy control, Epoxy with 30 ppm cavernamine-L, and Epoxy with 600 ppm cavernamine-L.

FIG. 14: Left: Gummi control, Right: Gummi with 180 ppm cavernamine-L.

### ABBREVIATIONS AND TERMS

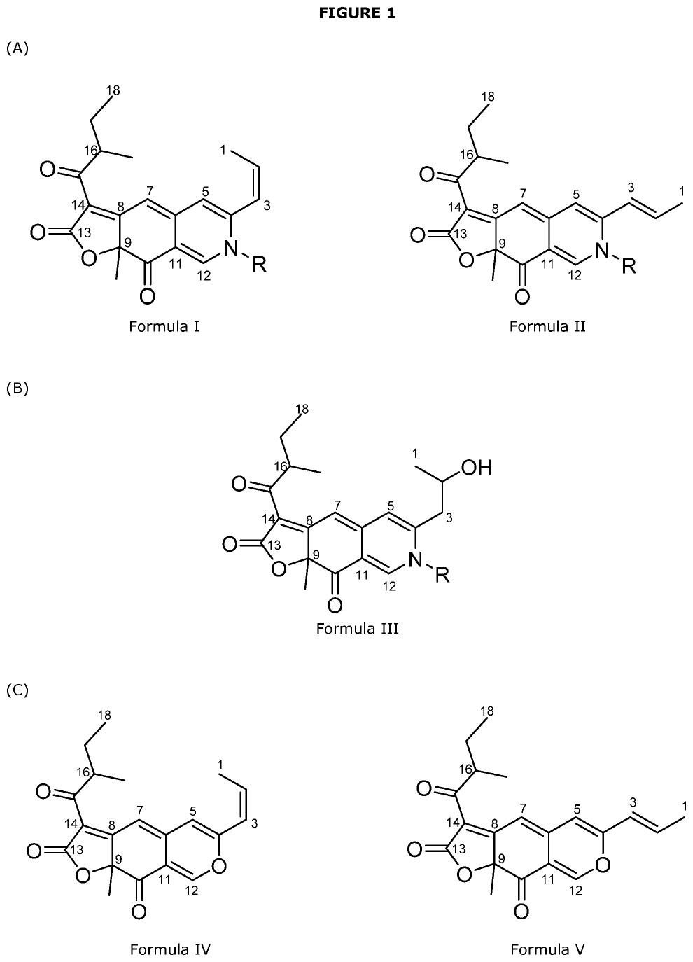

Cavernamine: is a pigment having the chemical formula C₂₀H₂₀O₄N—R (see formula I and II in FIG. 1). In the simplest cavernamine, R is hydrogen. In other cavernamine derivatives, N—R is a compound containing a primary amine, such as an amino acid, a peptide, an amino sugar.

Cavernamine amino acid derivative: is a cavernamine of the chemical formula C₂₀H₂₀O₄N—R, where N—R is an amino acid.

Hydroxyl-derivative of cavernamine: is used interchangeably with hydroxy-cavernamine; and has the chemical formula C₂₁H₂₁O₄N—R, where the carbon 2 has a hydroxyl group, and where N—R is an amino acid (see formula III in FIG. 1).

Cavernine: is a pigment having the chemical formula C₂₀H₂₀O₅ (see formula IV and V in FIG. 1); and is a precursor of cavernamine.

Growth medium essentially devoid of available inorganic nitrogen: is a growth medium which limits exponential growth and causes microbial (fungal) growth to enter a lag or cell death phase, due to lack of available nitrogen. The nitrogen source is depleted and no available nitrogen is left when the growth medium contains less than 5 mM of the nitrogen source (e.g. <5 mM KNO₃, NaNO₃, (NH₄)₂SO₄, or NH₄NO₃).

### DETAILED DESCRIPTION OF THE INVENTION

The present invention provides novel azaphilone pigments: cavernamines and carvernamine derivatives, as well as their precurser: cavernine. These red and orange/yellow pigments have potential use as e.g. food colorant. Further, a method for the production of individual species of azaphilone pigments by fermentation is provided, using fungal strains belonging to the species Aspergillus cavernicola. Strains of Aspergillus cavernicola were initially selected as a potential production organism since, in common with species of Monascus, they were found to excrete a bright red color when cultivated on solid media.

According to a first aspect, the invention provides a novel cavernamine pigment.

In one embodiment, the invention provides a novel cavernamine pigment having the formula I or formula II:

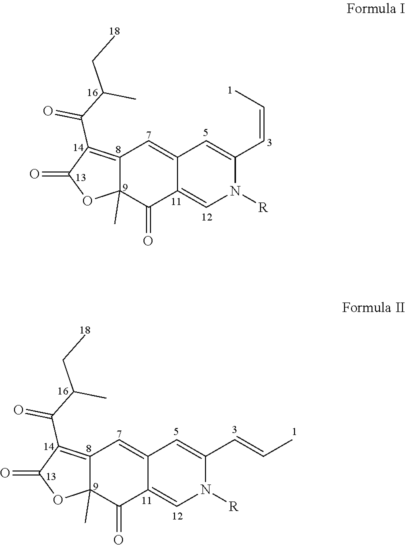

wherein R is hydrogen, or N—R is selected from among an amino acid, a peptide, an amino sugar (e.g. glucosamine or galactosamine) and a primary amine (e.g. anthranilic acid, aniline, ethanolamine or p-phenylenediamine).

In a further embodiment, the cavernamine pigment has formula I or II, wherein R is hydrogen.

In a preferred embodiment, the cavernamine pigment has formula I, wherein N—R is an amino acid. By way of example, the cavernamine pigment has formula I, wherein N—R is an amino acid selected from the group consisting of: L-alanine, L-arginine, L-asparagine, L-aspartate, L-cysteine, L-glutamine, L-glutamate, L-glycine, L-histidine, L-isoleucine, L-leucine, L-lysine, L-methionine, L-phenylalanine, L-proline, L-serine, L-threonine, L-tryptophan, L-tyrosine, L-valine and L-ornithine.

The novel cavernamine having formula I or II, as defined above, is a red azaphilone pigment naturally produced by Aspergillus cavernicola.

An important property of the novel cavernamine having formula I or II is its unexpected increased solubility in aqueous phase when compared to the known Monascus pigments (see Example 4). This may primarily be due to the shorter chain length of the backbone “tail” structure in the cavernamine.

According to a second aspect, the invention provides a novel hydroxy-cavernamine pigment.

In one embodiment, the invention provides a novel hydroxy-cavernamine pigment having the formula III:

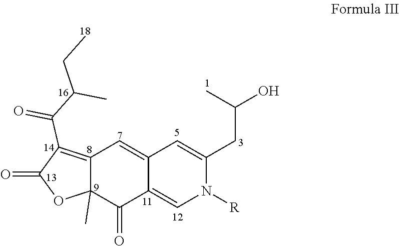

wherein R is hydrogen, or N—R is selected from among an amino acid, a peptide, an amino sugar (e.g. glucosamine or galactosamine) and a primary amine (e.g. anthranilic acid, aniline, ethanolamine or p-phenylenediamine).

In one embodiment, the hydroxy-cavernamine pigment has formula III, wherein R is hydrogen.

In a preferred embodiment, the hydroxy-cavernamine pigment has formula III, wherein N—R is an amino acid. By way of example, the hydroxy-cavernamine pigment has formula III, wherein N—R is an amino acid selected from the group consisting of: L-alanine, L-arginine, L-asparagine, L-aspartate, L-cysteine, L-glutamine, L-glutamate, L-glycine, L-histidine, L-isoleucine, L-leucine, L-lysine, L-methionine, L-phenylalanine, L-proline, L-serine, L-threonine, L-tryptophan, L-tyrosine, L-valine and L-ornithine.

The novel hydroxy-cavernamine having formula III, as defined above, is a red azaphilone pigment naturally produced by Aspergillus cavernicola.

Hydroxy-cavernamine is a hydroxyl-derivative of the carvernamine pigment of the present invention described above in the first aspect. Hence the core structure is the same (see FIG. 1, where only the arrangement of carbon 1-3 differs, while the core structure carbon 4-18 is identical) and which confer the improved technical properties observed.

An important property of the novel hydroxy-cavernamine having formula III is its increased solubility in aqueous phase when compared to the known Monascus pigments (see Example 4). This is primarily due to the shorter chain length of the backbone “tail” structure in the hydroxy-cavernamine as well as the hydroxyl-group in C2.

According to a third aspect, the invention provides a novel cavernine pigment.

In one embodiment, the invention provides a novel cavernine pigment having the formula IV or formula V:

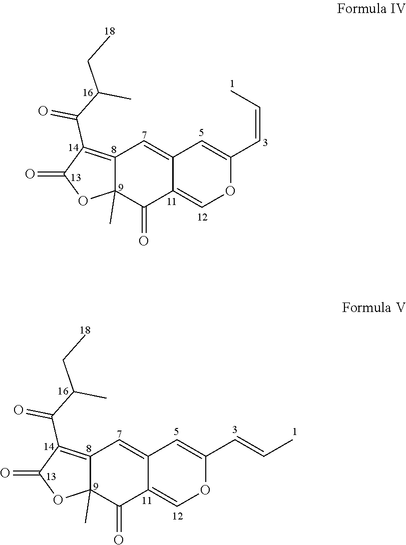 [SMILES: C/C=C\C1=CC2=CC3=C(C(=O)C(C)CC)C(=O)OC3(C)C(=O)C2=CO1 ; C/C=C/C1=CC2=CC3=C(C(=O)C(C)CC)C(=O)OC3(C)C(=O)C2=CO1]

The novel cavernine having formula IV or V, as defined above, is a yellow azaphilone pigment naturally produced by Aspergillus cavernicola.

Cavernine is a precursor of the carvernamine pigments of the present invention described above in the first and second aspects. Compared to carvernamine, cavernine has an oxygen atom instead of the N—R group. Hence the core structure is the same (see FIG. 1), which confers the improved technical properties observed.

An important property of the novel cavernine having formula IV or V is its increased water solubility when compared to the known Monascus pigments (see Example 4). This is primarily due to the shorter chain length of the backbone “tail” structure in the cavernamine.

Methods for extracting and detecting a cavernamine of formula I or II, a hydroxy-cavernamine of formula III or a carvenine of formula IV or V, according to a first, second and third aspect of the invention, are illustrated in Examples 1.4, 1.5 and 1.6. The chemical structure of a cavernamine of formula I or II, a hydroxy-cavernamine of formula III or a carvenine of formula IV or V, according to a first, second and third aspect of the invention, can be determined by means of Ultra-high Performance Liquid Chromatography coupled to Diode Array Detection and High Resolution Mass Spectrometry and Nuclear Magnetic Resonance (NMR) spectroscopy, as described in Examples 1.5 and 3.1.

A cavernamine of formula I or II, a hydroxy-cavernamine of formula III and/or a carvenine of formula IV or V, according to a first, second and third aspect of the invention can be used as a coloring agent in a food product, a non-food product and a cosmetic (such as described in Example 5). The food product may be selected from among the following foods: baked good, baking mix, beverage and beverage base, breakfast cereal, cheese, condiment and relish, confection and frosting, fat and oil, frozen dairy dessert and mix, gelatin, pudding and filling, gravy and sauce, milk product, plant protein product, processed fruit and fruit juice, and snack food.

The non-food product may be selected from among the following non-foods: textile, cotton, wool, silk, leather, paper, paint, polymer, plastic, and inks.

The cosmetic product may be in the form of a free, poured or compacted powder, a fluid anhydrous greasy product, an oil for the body and/or the face, a lotion for the body and/or the face, or a hair product.

The invention further provides a kit of parts for coloring a composition, wherein the kit comprises at least (i) one cavernamine pigment having formula I or II, at least one hydroxy-cavernamine of formula III and/or at least one carvenine of formula IV or V according to the invention and (ii) a stabilizing agent, wherein the composition is selected from among a food, a non-food product and a cosmetic. The stabilizing agent may be gum arabic or similar food industry stabilizer. The kits of part may further comprise maltodextrin or other food additives with properties similar to maltodextrin.

An example of such composition is provided in Example 6. The pigment is preferably supplied in a container (optionally combined with a dispensing agent e.g. colloid or thickening agent).

According to a fourth aspect, the invention provides a method for producing cavernamine pigments and/or their hydroxyl-derivatives.

According to one embodiment, the invention provides a (1-step) method for producing cavernamine pigment and/or hydroxyl-derivative of said cavernamine pigment by fermentation comprising the steps of:
a) providing spores or mycelia of a strain of Aspergillus cavernicola,
b) cultivating said spores or mycelia in a liquid growth medium comprising a nitrogen source,
c) recovering the cavernamine pigment and/or hydroxyl-derivative of said cavernamine pigment produced during cultivation in step (b), and
d) optionally isolating one or more of said cavernamine pigments and/or hydroxyl-derivative of said cavernamine pigment,
wherein said cavernamine pigment has the structure of Formula I or II

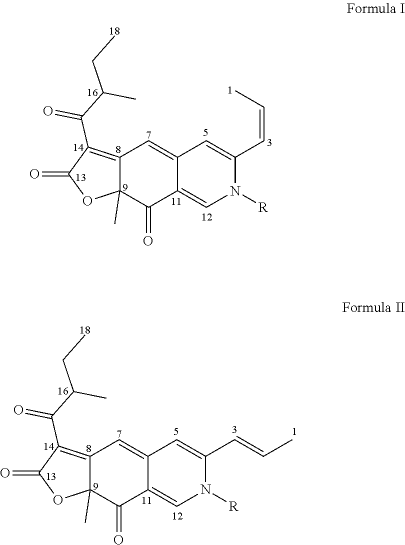

and wherein said hydroxyl-derivative of said cavernamine pigment has the structure of formula III:

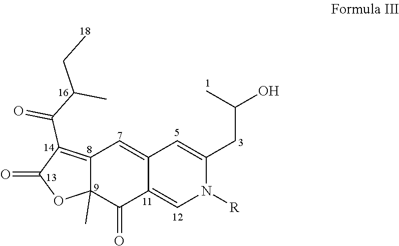

In one embodiment, the nitrogen source of the liquid growth medium is selected from a complex source such as yeast extract or corn steep liquor. In another embodiment, the nitrogen source may be urea. In yet another embodiment, the nitrogen source is selected from an inorganic nitrogen source such as KNO₃, NaNO₃, (NH₄)₂SO₄, or NH₄NO₃.

In a preferred embodiment, the nitrogen source in the liquid growth medium in step (b) solely consists of a compound selected from the group consisting of an amino acid, a peptide, an amino sugar and any other primary amine.

A suitable sole nitrogen source includes an amino sugar such as glucosamine or galactosamine; and includes a primary amine such as anthranilic acid, aniline, ethanolamine or p-phenylenediamine.

Even more preferably, the sole nitrogen source is a single amino acid, selected from one of the group consisting of: L-alanine, L-arginine, L-asparagine, L-aspartate, L-cysteine, L-glutamine, L-glutamate, L-glycine, L-histidine, L-isoleucine, L-leucine, L-lysine, L-methionine, L-phenylalanine, L-proline, L-serine, L-threonine, L-tryptophan, L-tyrosine, L-valine and L-ornithine.

The liquid growth medium, comprising a nitrogen source, is preferably a synthetic medium comprising salts, trace metals, and a source of carbon. A suitable source of carbon includes glucose, sucrose, maltose, soluble starch, beet or cane molasses, malt and any combination of at least two thereof.

The growth medium preferably further comprises or consists of the following salts and trace metals: KH₂PO₄ (for example 1 g/L), NaCl (for example 1 g/L), MgSO₄.7H₂O (for example 2 g/L), KCl (for example 0.5 g/L), CaCl₂.H₂O (for example 0.1 g/L) and a trace metal solution (for example 2 mL/L). The trace metal solution may comprise, or consist, of: CuSO₄.5H₂O (for example 0.4 g/L), Na₂B₄O₇.10H₂O (for example 0.04 g/L), FeSO₄.7H₂O (for example 0.8 g/L), MnSO₄.H₂O (for example 0.8 g/L), Na₂MoO₄.2H₂O (for example 0.8 g/L), ZnSO₄.7H₂O (for example 8 g/L).

The concentration of the compound providing the nitrogen source in the growth medium may be from 0.01M to 1M, for example at least 0.01, 0.025, 0.05, 0.075, 0.10, 0.125, 0.15, 0.175, 0.2, 0.3, 0.4, 0.5, 0.6, 0.7, and 0.8M.

The pH of the growth medium provided and maintained during step (b) is preferable between 3 and 8, more preferably between 4.0 and 6.5, even more preferably between 4.0 and 6.0; where the pH may be adjusted by the addition of aqueous NaOH or HCl.

Cultivation in step (b) may be performed by suspending spores or mycelia of Aspergillus cavernicola in the liquid growth medium.

The spores in step (a) may comprise an aqueous suspension of spores of Aspergillus cavernicola.

In one embodiment, the cavernamine pigment and/or its hydroxyl-derivative produced according to the 1-step method of the invention has the structure of Formula I or III, wherein N—R is selected from among an amino acid, a peptide, an amino sugar and a primary amine.

According to a second embodiment, the invention provides a (2-step) method for producing a cavernamine pigment of Formula I and/or a hydroxyl-cavernamine of Formula III using a modification of the 1-step fermentation procedure described above. According to this modification, an additional step (a′) is performed after step (a). In step (a′), the spores or mycelia provided in step (a) are cultivated in a preliminary liquid growth medium, wherein the sole nitrogen source is urea or an inorganic nitrogen source. The inorganic nitrogen source may be selected from the group consisting of: KNO₃, NaNO₃, (NH₄)₂SO₄, and NH₄NO₃.

Preferably, the concentration of the nitrogen source in the preliminary growth medium is less than 50 mM, such as no more than 45, 40, 35, 30, 25, 20, 17.5, 15, 12.5, or 10 mM

The preliminary liquid growth medium in step (a′), comprising the inorganic nitrogen as sole nitrogen source, is a synthetic medium comprising salts, trace metals, and a source of carbon. A suitable source of carbon includes glucose, sucrose, maltose, soluble starch, beet or cane molasses, malt and any combination of at least two thereof. The composition of this synthetic medium with respect to salts and trace metals preferably comprises or consiss of: KH₂PO₄ (for example 1 g/L), NaCl (for example 1 g/L), MgSO₄.7H₂O (for example 2 g/L), KCl (for example 0.5 g/L), CaCl₂.H₂O (for example 0.1 g/L) and a trace metal solution (for example 2 mL/L). The trace metal solution may comprise, or consist of: CuSO₄.5H₂O (for example 0.4 g/L), Na₂B₄O₇.10H₂O (for example 0.04 g/L), FeSO₄.7H₂O (for example 0.8 g/L), MnSO₄.H₂O (for example 0.8 g/L), Na₂MoO₄.2H₂O (for example 0.8 g/L), ZnSO₄.7H₂O (for example 8 g/L.

According to the 2-step fermentation method, cultivation of the Aspergillus culture produced in step (a′) is then continued with a further cultivation step (b) in a liquid growth medium. The liquid growth medium in step (b) is preferably a synthetic medium having the same composition with respect to salts and trace metals as the preliminary liquid growth medium. However, the liquid growth medium in step (b) additionally comprises a source of organic nitrogen. Suitable organic nitrogen sources are selected from the group consisting of an amino acid, a peptide, an amino sugar and any other primary amine; and correspond to suitable sources used in the liquid growth medium in the 1-step fermentation procedure. The organic nitrogen compound is preferably selected from one of an amino acid, a peptide, an amino sugar and a primary amine as a sole source of organic nitrogen.

Although a source of inorganic nitrogen is a component of the preliminary liquid growth medium in step (a′); no additional source of inorganic nitrogen is included in the liquid growth medium in step (b), but instead the inorganic nitrogen is substituted with the given sources of organic nitrogen.

2-step fermentation, according to the second embodiment, may be performed by cultivating the spores or mycelium in the preliminary liquid growth medium in step (a′), and then adding in step (b) the sole source of organic nitrogen to the culture produced by step (a′). The inorganic nitrogen content of the preliminary liquid growth medium is depleted during cultivation of the fungal spores or mycelium in step (a′), such that the growth medium is essentially devoid of available inorganic nitrogen at the end of step (a′). The inorganic nitrogen content of the preliminary liquid growth medium can be adjusted to ensure complete depletion by the end of step (a′); for example by providing no more than 50 mM, 45 mM, 40 mM, 35 mM, 30 mM, 25 mM, 20 mM, 17.5 mM, 15 mM, 12.5 mM, 10 mM of NO₃⁻ or NH₄⁺. Once the level of inorganic nitrogen present in the preliminary liquid growth medium is depleted to an amount of less than 5 mM, 4 mM, 3 mM, 2 mM, 1 mM, 0.5 mM of NO₃⁻ or NH₄⁺, then it is no longer able to support growth of the Aspergillus culture.

Alternatively, the preliminary liquid growth medium in step (a′) is replaced by the liquid growth medium comprising the above identified organic nitrogen compound as sole nitrogen source, at the start of the further cultivation step (b).

The pH of the preliminary growth medium provided in step (a′) may be the same or different from the pH of the growth medium in step (b).

The pH of the preliminary growth medium provided and maintained during step (a′) is preferable between 3 and 8, such as between 3 and 5, such as between 4 and 7, more preferably between 4.0 and 6.5, even more preferably between 4.0 and 6.0; where the pH may be adjusted by the addition of aqueous NaOH or HCl.

The pH of the growth medium provided and maintained during step (b) is preferable between 3 and 8, more preferably between 4.0 and 6.5, even more preferably between 4.0 and 6.0; where the pH may be adjusted by the addition of aqueous NaOH or HCl.

The cavernamine pigment and/or its derivative produced according to the 2-step method of the invention has the structure of Formula I or III, wherein N—R is selected from among an amino acid, a peptide, an amino sugar and a primary amine.

The cultivation conditions during 1-step and 2-step fermentation support aerobic metabolism in the Aspergillus culture. Aerobic metabolism relies on a sufficient aeration, which can be achieved by shaking the liquid culture or by supplying a source of air (e.g. oxygen).

The 1-step and 2-step fermentation procedure can be performed in a bioreactor. The liquid growth media (described above) used in both the 1-step and 2-step fermentation procedure may be supplied to the bioreactor to facilitate either batch, fed-batch or continuous culture of the fungal culture.

The duration of the cultivation steps (a′) and (b) in the 2-step fermentation procedure are selected to optimise growth of the Aspergillus culture (as measured by biomass) and the yield of pigment produced by the Aspergillus culture. The cultivation step (a′) is preferably at least 28 h; for example between 30 h and 40 h. The cultivation step (a′) may be about 32, 34, 36, 38, 40, 42, 44, 46, 48, 50, 52, 54, 56, 58, 60, 62, 64, 66, 68, 70, and 72 h in duration. The duration of the cultivation step (b), that follows step (a′), is preferably at least 48 h, at least 72 h, at least 96 h, or even at least 120 h. The cultivation step (b) may for example be between 48 h and 168 h. The cultivation step (b) may be about 48, 54, 60, 66, 72, 78, 84, 90, 96, 104, 110, 116, 120, 144, or even 168 h in duration.

The cavernamine and hydroxy-carvernamine pigments produced by the cultivation of Aspergillus cavernicola is extracellular and can therefore be recovered from the liquid medium.

Surprisingly, the red pigment produced by the 2-step method of the invention is essentially a single species of cavernamine and hydroxy-carvernamine pigment and not a mixture of pigments (see Example 1). When low amounts of inorganic nitrogen source is supplied during step (a′) of the 2-step fermentation procedure, this selectively promotes the synthesis of low amounts of both cis- and trans-forms of the yellow/orange cavernine pigment of Formula IV and V, respectively, during step (a′). In subsequent step (b), the amino-group present in the source of organic nitrogen is incorporated into the cavernine core isomeric structures (cis- and trans) to form the specific cis-cavernamine derivative of Formula I in essentially pure form. Thus the single species of cavernamine pigment produced by the method can be extracted and recovered without the need for multiple and possibly complex purification steps. Furthermore, the products of the fermentation using the method are free of any mycotoxin (see Example 2), and are therefore safe for human use.

According to a fifth aspect, the invention provides a method for producing cavernine pigments.

According to one embodiment, the invention provides a method for producing a cavernine pigment by fermentation comprising the steps of:
a) providing spores or mycelia of a strain of Aspergillus cavernicola,
b) cultivating said spores or mycelia in a liquid growth medium,
c) recovering the cavernine pigment produced during cultivation in step (b), and
d) optionally isolating said cavernine pigment,
wherein said cavernine pigment has the structure of Formula IV or V:

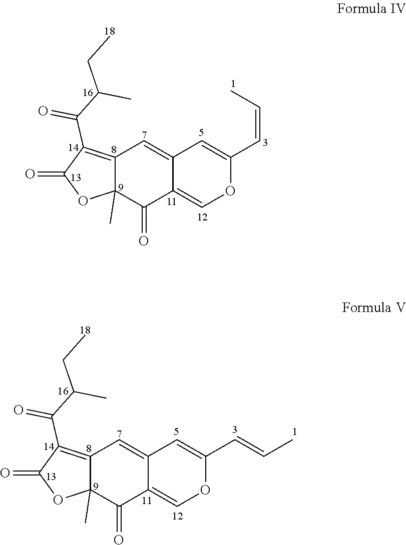 [SMILES: C/C=C\C1=CC2=CC3=C(C(=O)C(C)CC)C(=O)OC3(C)C(=O)C2=CO1 ; C/C=C/C1=CC2=CC3=C(C(=O)C(C)CC)C(=O)OC3(C)C(=O)C2=CO1]

For cavernine production, the spores or mycelia provided in step (a) are in step (b) cultivated in a liquid growth medium, wherein the nitrogen source may be urea or a complex nitrogen source such as yeast extract or corn steep liquor, or the nitrogen source may be an inorganic nitrogen source, such as selected from the group consisting of: KNO₃, NaNO₃, (NH₄)₂SO₄, and NH₄NO₃.

Preferably, the concentration of the nitrogen source in the growth medium for cavernine production is less than 50 mM, such as no more than 45, 40, 35, 30, 25, 20, 17.5, 15, 12.5, or 10 mM.

The liquid growth medium may be a synthetic medium comprising salts, trace metals, and a source of carbon. A suitable source of carbon includes glucose, sucrose, maltose, soluble starch, beet or cane molasses, malt and any combination of at least two thereof. The composition of this synthetic medium with respect to salts and trace metals preferably comprises or consiss of: KH₂PO₄ (for example 1 g/L), NaCl (for example 1 g/L), MgSO₄.7H₂O (for example 2 g/L), KCl (for example 0.5 g/L), CaCl₂.H₂O (for example 0.1 g/L) and a trace metal solution (for example 2 mL/L). The trace metal solution may comprise, or consist of: CuSO₄.5H₂O (for example 0.4 g/L), Na₂B₄O₇.10H₂O (for example 0.04 g/L), FeSO₄.7H₂O (for example 0.8 g/L), MnSO₄.H₂O (for example 0.8 g/L), Na₂MoO₄.2H₂O (for example 0.8 g/L), ZnSO₄.7H₂O (for example 8 g/L.

Fermentation for production of cavernine, according to the fifth embodiment, may be performed in a bioreactor, such as run in batch, fed-batch or continuous mode. The nitrogen content of the liquid growth medium in step (b) may be depleted during fermentation such that the growth medium is essentially devoid of available nitrogen at the end of step (b); or a supply of nitrogen source (possibly mixed with other medium components/nutrients) may be supplied during step (b) to provide a minimum nitrogen concentration to sustain the cells. The nitrogen content of the liquid growth medium in step (b) can be adjusted initially, throughout, or at certain intervals to be 50 mM, 45 mM, 40 mM, 35 mM, 30 mM, 25 mM, 20 mM, 17.5 mM, 15 mM, 12.5 mM, or 10 mM of nitrogen source, such as 50 mM, 45 mM, 40 mM, 35 mM, 30 mM, 25 mM, 20 mM, 17.5 mM, 15 mM, 12.5 mM, or 10 mM NO₃⁻ or NH₄⁺.

Cultivation time in step (b) should preferably be adjusted to avoid the potential onset of cavernamine production. Such adjustment may involve terminating cultivation after 16 h, 20 h, 24 h, 28 h or 32 h; for example between 20 h and 46 h. The cultivation step (b) may be about 32, 34, 36, 38, 40, 42, 44, 46, 48, 50, 52 and 54 h in duration.

The pH of the growth medium provided and maintained during step (b) is preferable between 3 and 8, such as between 3 and 5, such as between 4 and 7, more preferably between 4.0 and 6.5, even more preferably between 4.0 and 6.0; where the pH may be adjusted by the addition of aqueous NaOH or HCl.

The cavernine pigments produced by cultivation of Aspergillus cavernicola is extracellular and can therefore be recovered from the liquid medium.

### EXAMPLES

### Example 1. Production of Cavernamines by Fermentation

### 1.1 Strain Maintenance and Spore Production:

The fungal strains, Aspergillus cavernicola IBT 32660 and IBT 23158 (IBT Technical University of Denmark strain collection), were used for production of cavernines and cavernamines. Spores of A. cavernicola were propagated on plates on CYA agar (Czapek Dox Yeast extract Agar supplied by Sigma-Aldrich) and incubated at 25° C. for 7 days. Spores were harvested with 0.9% sodium chloride (NaCl) solution and 0.01% Tween 20; the suspension was filtered through mira-cloth to separate spores from mycelia. The spore solution was centrifuged for 10 min at 10.000 rpm at 4° C. The supernatant was removed and the spore pellet was re-suspended in 0.9% NaCl solution. The spore concentration was determined by using a Burker-Turk counting chamber. All cultivations were inoculated in a specified medium to give an initial spore concentration of 10⁶ spores/ml.

### 1.2 Sampling

Samples for dry weight (DW), HPLC, absorbance and LC-MS analysis were taken at the end of shake flask cultivation or regularly throughout the cultivations in bioreactors. Samples intended for HPLC, absorbance and LC-MS were filtered through a sterile filter with a pore size of 0.45 μm in order to separate biomass from the filtrate.

1.3 Dry Weight Analysis: Analysis of A. cavernicola Biomass Obtained by Fermentation

Dry weight (DW) was assessed on filters which were pre-dried in a microwave for 20 min, kept in a desiccator for a minimum of 10 min and weighed. For DW analysis, the filters were placed in a vacuum filtration pump and 10 ml of culture broth was added. Subsequently the filters with the biomass were dried in a microwave for 20 min and kept in a desiccator for a minimum of 10 min before being re-weighed. The weight of the biomass was determined as the difference of the filter weight before and after sample application.

### 1.4 Extraction and Purification

Pigments were extracted from submerged cultivation of A. cavernicola by first separating biomass and media by filtration. Next, the media was extracted using ethyl acetate and the ethyl acetate phase was dried. The dried extract was fractionated on an Isolera One (Biotage) flash system equipped with a diol column, using n-heptane, n-heptane:dichloromethane (1:1), dichloromethane, dichloromethane:ethyl acetate (1:1), ethyl acetate, ethyl acetate:methanol (1:1), and methanol. The fractions containing the pigments were further subjected to semi-preparative HPLC on a Waters 600 Controller connected to a Waters 966 PDA detector. The column used was a Phenomenex Luna II C18, and the compounds were eluted using a gradient of MQ water and acetonitrile with 50 ppm triflouroacetic acid.

### 1.5 Ultra-High Performance Liquid Chromatography-High Resolution Mass Spectrometry (UHPLC-HRMS)

UHPLC-HRMS was performed on an Agilent Infinity 1290 UHPLC system (Agilent Technologies, Santa Clara, Calif., USA) equipped with a diode array detector. Separation was obtained on an Agilent Poroshell 120 phenyl-hexyl column (2.1×250 mm, 2.7 μm) with a linear gradient consisting of water (A) and acetonitrile (B) both buffered with 20 mM formic acid, starting at 10% B and increased to 100% in 15 min where it was held for 2 min, returned to 10% in 0.1 min and remaining for 3 min (0.35 mL/min, 60° C.). An injection volume of 1 μL was used. UV-VIS detection was done on an Agilent 1290 DAD detector with a 60 mm flowcell. MS detection was performed in positive detection mode on an Agilent 6545 QTOF MS equipped with Agilent Dual Jet Stream electrospray ion source with a drying gas temperature of 250° C., gas flow of 8 L/min, sheath gas temperature of 300° C. and flow of 12 L/min. Capillary voltage was set to 4000 V and nozzle voltage to 500 V. Mass spectra were recorded at 10, 20 and 40 eV as centroid data for m/z 85-1700 in MS mode and m/z 30-1700 in MS/MS mode, with an acquisition rate of 10 spectra/s. Lock mass solution in 70:30 methanol:water was infused in the second sprayer using an extra LC pump at a flow of 15 μL/min using a 1:100 splitter. The solution contained 1 μM tributylamine (Sigma-Aldrich) and 10 μM Hexakis(2,2,3,3-tetrafluoropropoxy)phosphazene (Apollo Scientific Ltd., Cheshire, UK) as lock masses. The [M+H]⁺ ions (m/z 186.2216 and 922.0098 respectively) of both compounds was used.

### 1.6 Absorbance Analysis: Quantitative Analysis of Carvernamines Produced by Fermentation

Quantitative analysis of pigments was performed by absorbance measurements. Absorbance values of the individual pigment solutions were determined using a Synergy 2 photo spectrum (BioTek, Germany) and a 96 well microtiter plate. 150 μL of sample broth of each amino-acid-pigment-solution were scanned in the range of 200-700 nm and maximum absorbance values were determined. Absorbance at 500 nm indicated presence of red pigments. A standard curve of an orange and red pigment was used to calculate the concentration in the medium. For the amino acids, where no standard curve was available the absorbance is given in AU/150 μL.

### 1.7 Initial Screening: One-Step Fermentation Procedure for Production of Cavernamines

Initial screening of the two strains was conducted (i) on Czapek Yeast Extract Agar (CYA) plates as well as (ii) in liquid Czapek Dox broth.

(i) A. cavernicola spores were propagated on CYA plates incubated at 25° C. for 7 days. Plug extractions were performed by taking 3-5 plugs of 6 mm diameter across a colony. The plugs were transferred to Eppendorf tubes and extracted with 800 μL of a 3:1 mixture of ethyl acetate and iso-propanol, with 1% (v/v) formic acid (FA), for one hour with sonication. Following sonication, the extraction liquid was decanted to new Eppendorf tubes, and the solvent was evaporated under a gentle stream of nitrogen gas at 30° C. The dried extracts were re-dissolved in 400 μL methanol (MeOH) with sonication, and centrifuged for 3 min at 13500 rpm to avoid any spores or other particles in the sample. The chromatographic profile of the extracellular compounds secreted by A. cavernicola was prepared as described in example 1.5.

(ii) A. cavernicola spores were inoculated in Czapek Dox broth (pH 6) and cultured for 7 days. Czapek Dox broth consisted of sucrose (30 g/L), NaNO3 (3 g/L), MgSO4.7H2O (0.5 g/L), KCl (0.5 g/L), K2HPO4 (1 g/L), FeSO4 (0.01 g/L)), and 1 ml/L trace metal solution. The trace metal solution consisted of CuSO4.5H2O (0.5 g/L), and ZnSO4.7H2O (1 g/L). Cultivation was carried out in non-baffled shakeflasks at 25° C. and 150 RPM (Forma orbital shaker, Thermo FIsher Scientific, US) with a sample volume of 100 ml. Shake flask experiments were carried out in duplicates. Samples were taken after 7 days. The chromatographic profile of the extracellular compounds secreted by A. cavernicola was prepared as described in example 1.5.

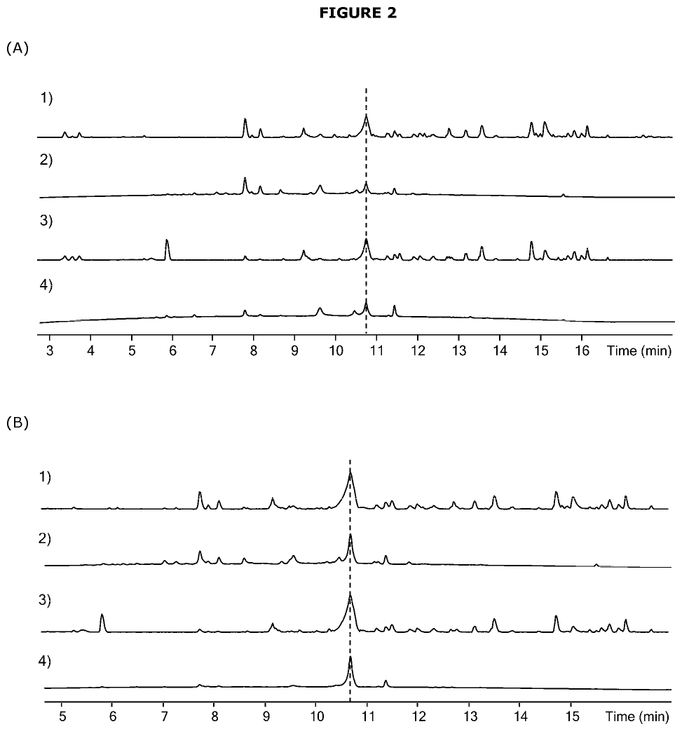

It was visually observed that both the plates as well as the liquid culture medium turned red during cultivation of A. cavernicola. The chromatographic profile of extracellular compounds secreted by A. cavernicola are seen in FIG. 2, showing a wide range of pigments produced. The metabolic profiles from CYA plates and Czapek Dox broth have similar peaks. It was thereby demonstrated that they can be equally used for subsequent testing of cavernamine production by A. cavernicola.

### 1.8 Initial Screening: Two-Step Fermentation Procedure for Production of Cavernamines

A. cavernicola spores were inoculated in Czapek Dox broth (pH 6) consisting of sucrose (30 g/L), NaNO3 (3 g/L), MgSO4.7H2O (0.5 g/L), KCl (0.5 g/L), K2HPO4 (1 g/L), FeSO4 (0.01 g/L)), and 1 ml/L trace metal solution. The trace metal solution consisted of CuSO4.5H2O (0.5 g/L), and ZnSO4.7H2O (1 g/L). Additional nitrogen source in the form of amino acids (e.g. L-leu, L-his, L-val, L-arg, or L-trp) was added in a concentration of 2 mM after 5 days of cultivation. Cultivations were carried out in baffled shakeflasks at 25° C. and 150 RPM (Forma orbital shaker, Thermo Fisher Scientific, US) with a sample volume of 100 ml. Shake flask experiments were carried out in duplicates. Czapek Dox broth without addition of amino acids was used as control/benchmark (Example 1.7(ii)). Samples were taken after 7 days. The chromatographic profile of the extracellular compounds secreted by A. cavernicola was prepared as described in example 1.5.

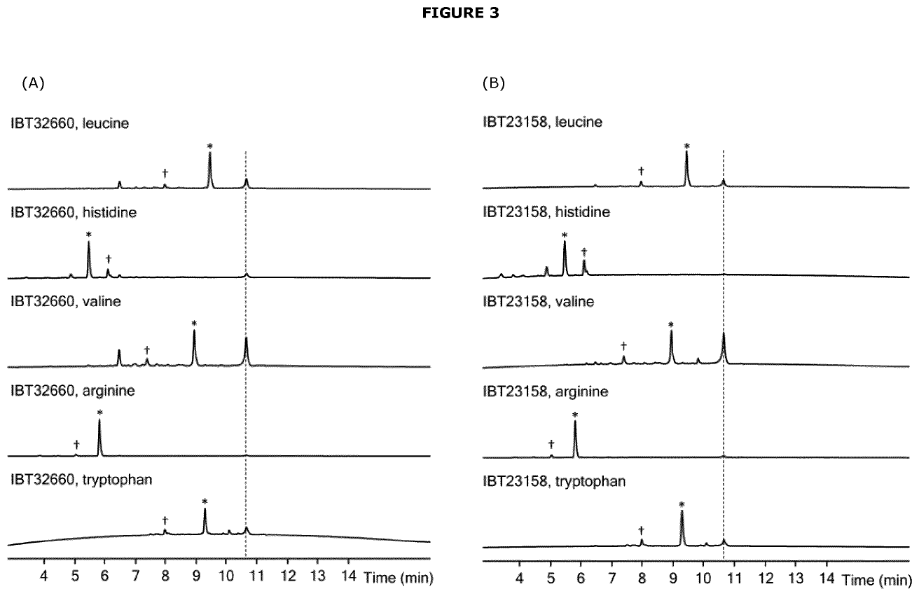

The chromatographic profile of the amino acid induced cultures showed a significantly leaner profile (FIG. 3) compared to the non-induced samples (FIG. 2). The cavernamine amino acid derivatives were found to be the major constituent of the broth of the amino acid induced samples.

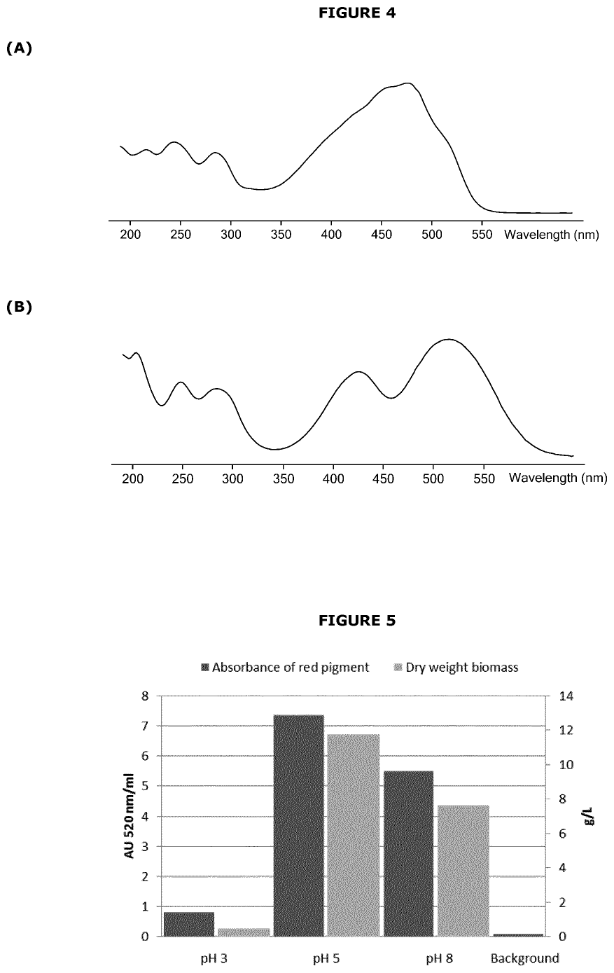

Absorbance spectra of cavernine and cavernamine (exemplary cavermanine-L) are presented in FIG. 4.

### 1.9 pH Screening for Cavernamine Production

Aspergillus cavernicola IBT 32660 was cultured in liquid Czapek dox broth (35 g/L) supplemented with yeast extract (5 g/L) and 1 ml/L of trace metal solution consisting of CuSO₄.5H₂O (0.5 g/L), and ZnSO₄.7H₂O (1 g/L). The pH was adjusted by KOH or H₂SO₄ to pH 3, 5, and 8. Cultivations were run for 168 hours in shake flasks, with a sample volume of 50 ml at 25° C., 150 rpm. Pigment production was assessed at the end of cultivation by absorbance analysis. The culture media was filtered through a 0.45 μm pore size filter, and absorbance measured at 520 nm in a spectrophotometer. HPLC-MS analysis as described in example 1.5 was conducted on all three samples; and dry weight analysis as described in example 1.3 was also performed.

Results of the pH screening are presented in FIG. 5, showing that production of cavernamine is possible at a pH range between 3-8, however pH 5 is much preferred. At pH 3 growth of the fungus is very inhibited and slow, which most likely explains the low amounts of pigments produced.

### Example 2. Products of A. cavernicola are Free of the Mycotoxin Citrinin

Analysis (as described in example 1.5) of extracts derived from A. carvernicola cultivated on CYA (5 g/l yeast extract, 35 g/l Czapek dox broth, 20 g/l agar, 1 ml/l trace metals), MEA (20 g/l malt extract, 1 g/l peptone, 20 g/I glucose, 20 g/l agar, 1 ml/l trace metals), OAT (30 g/l oat meal, 15 g/l agar, 1 ml/l trace metals), PDA (39 g/l potato dextrose agar, 1 ml/l trace metals) and YES (20 g/l yeast extract, 150 g/l sucrose, 0.5 g/l MgSO4/H2O, 1 ml/l trace metals) shows that the mycotoxin citrini is not produced (data not shown) under any of the cultivation conditions.

### Example 3. Structure of Novel Cavernamine, Cavernine, and Hydroxy-Carvernamine Pigments Produced by Fermentation of A. cavernicola

From cultivations of A. cavernicola, a total of four different kinds of novel azaphilone compounds were identified: Cavernines, cavernamines, amino acid derivatives of cavernamines, and hydroxy-derivatives of cavernamines.

Structures of cavernine, cavernamine, amino acid derivatives of cavernamines, and hydroxy-cavernamines were determined using 1D and 2D NMR experiments. A. cavernicola pigments were extracted, separated and analysed as described in Example 1.4 and 1.5; and subsequently analysed using NMR as described below:

### 3.1 Nuclear Magnetic Resonance (NMR) Spectroscopy

NMR spectra (1H, DQF-COSY, edHSQC, HMBC and NOESY) were recorded on a Bruker Avance 800 MHz located at the Department of Chemistry at the Technical University of Denmark. NMR spectra were acquired using standard pulse sequences. The solvent used was either DMSO-d6, which was also used as reference with signals at δH=2.50 ppm and δC=39.5 ppm, or CD₃OD (reference at δH=3.31 ppm and δC=49.0 ppm). Data processing and analysis was done using TopSpin 3.5 (Bruker), MestReNova v. 6.2.1-7569 (Mestrelab Research, Santiago de Compostela, Spain) and ACD NMR Workbook (Advanced Chemical Development, Inc., Toronto, Ontario, Canada). J-couplings are reported in hertz (Hz) and chemical shifts in ppm (0).

### 3.2 Structural Elucidation of Cavernamines

Based on HR-MS, the formula of the two isomers of cavenamine was determined to be C₂₀H₂₁NO₄ (measured m/z of [M+H]⁺=340.1541).

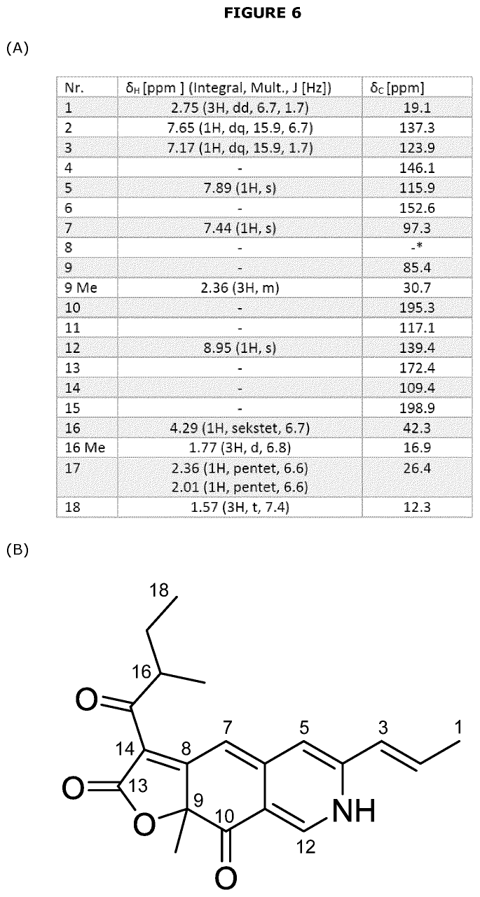

From the 1H spectrum, 21 protons were identified, along with 19 carbons based on the HSQC and HMBC, listed in FIG. 6A. The apparent absence of one carbon signal is in agreement with previously obtained results from other azaphilone compounds, as carbon 8 (FIG. 6B) often has a low signal intensity when spectra are acquired in methanol.

The DQF-COSY spectrum showed correlations between the protons at C-1, C-2 and C-3, as well as between H-16, H-16-CH₃, H-17, and H-18. The remaining part of the structure was determined using HMBC correlations. The protons H-3, H-5, and H-12 showed correlations to the quaternary C-4, while the protons H-5 and H-12 had additional correlations to C-6 and C-11. C-4 and C-12 were determined to be placed on either side of a heteroatom, specifically a nitrogen. H-7 had correlations to C-5, C-6, and C-11. In addition, a correlation to the ketone C-10 was observed from H-12 and H-9-CH₃. C-9 showed correlations to the methyl group C-9-CH₃, which further had correlations to the carbonyl C-13, determined to be part of a lactone. The protons on C-16, C-16-CH₃, and C-17 all had correlations to the ketone C-15.

Based on the observed correlations, a central heteroaromatic bicyclic structure (C-4 to C-12) linked to a lactone was established. An aliphatic moiety consisting of four carbons (C-16, C-16-CH₃, C-17, and C-18) could be attached to the lactone part (C-13 and C-14) via C-15. A single methylation was determined to be placed at C-9, while a short three-carbon chain (C-1 to C-3) containing a single double bond was found to be linked to the heteroaromatic part at C-4. Based on the coupling constant shared between H-2 and H-3, the double bond was determined to be in a trans-configuration. The structure of the compounds which has been named trans-cavernamine is shown in FIG. 6B.

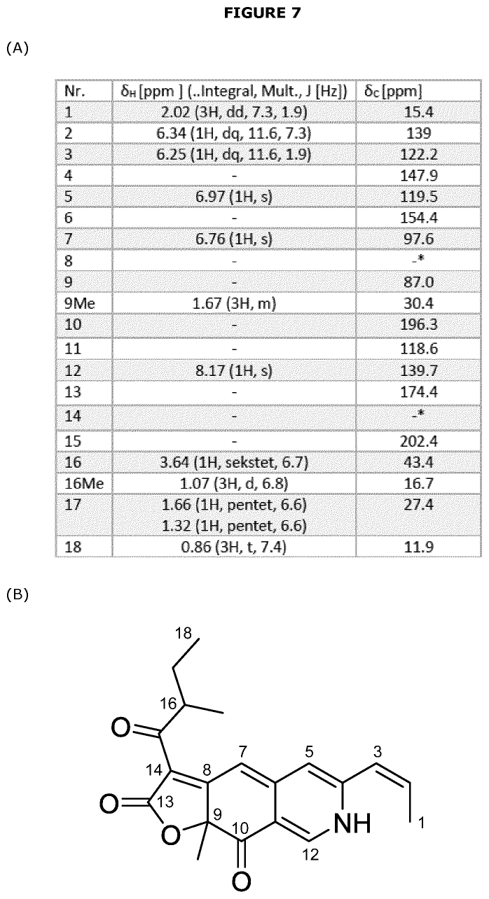

In addition to the trans-version of cavernamine, a cis-version was also isolated (FIG. 7B). The chemical shifts were highly comparable to the trans-version, with differences being mainly in at H-2 and H-3, for which the coupling constants corresponded to the cis-configuration (FIG. 7A).

### 3.3 Structural Elucidation of Cavernamine Amino Acid Derivatives

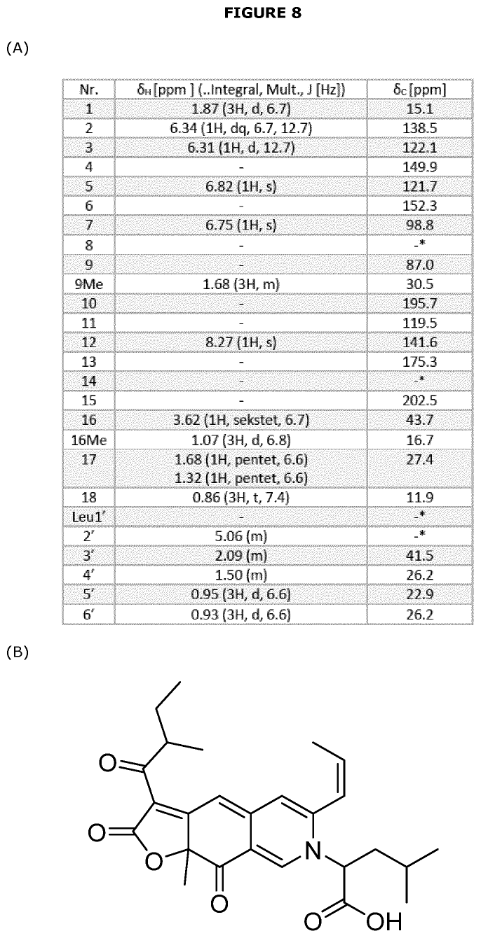

Amino acid derivatives of cavernamines obtained from the shake flask cultivations described in example 1.8 were isolated and structurally elucidated. Each of the derivatives are named according to the incorporated amino acid. As an example, FIG. 8A lists proton and carbon shifts for the leucine derivative, cis-cavernamine-L (FIG. 8B).

### 3.4 Structural Elucidation of Cavernines

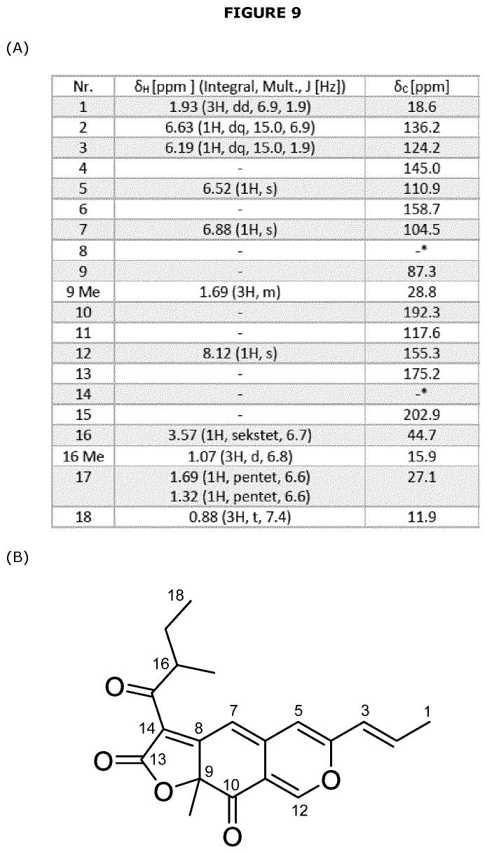

In addition to the nitrogen containing cavernamines, orange/yellow pigments not containing nitrogen were isolated from shake flask cultivations prior to addition of amino acids (FIG. 9B). HR-MS analysis determined the formula to be C₂₀H₂₀O₅. The chemical shifts were highly similar to those for cavernamines and can be found in FIG. 9A.

### 3.5 Structural Elucidation of Hydroxy-Cavernamines

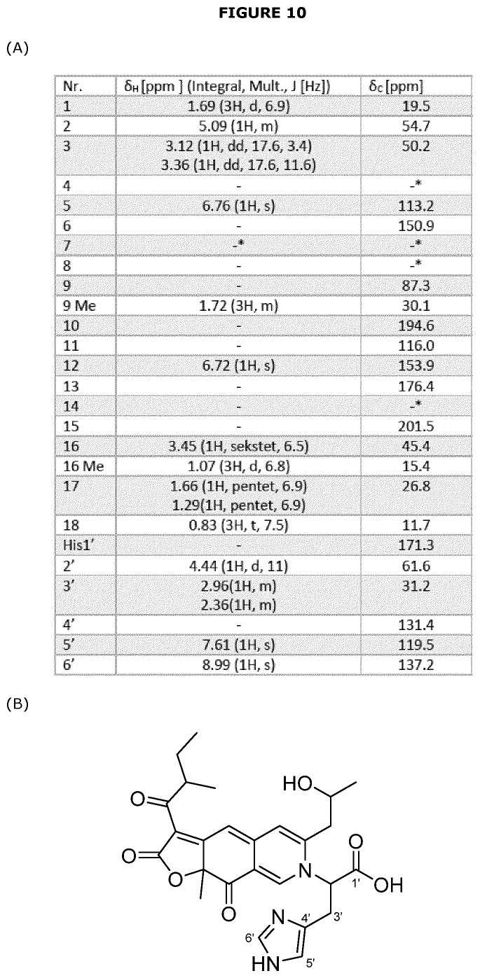

A series of less reduced amino acid containing cavernamines were also identified from the shake flask cultivations described in example 1.8, containing a hydroxyl group at C-2 instead of the double bond between C-2 and C-3 (FIG. 10B). As an example, NMR data for the histidine derivative, hydroxy-cavernamine-H is found in FIG. 10A.

### Example 4. Physical Properties of Cavernamines Pigments

Based on calculations (http://www.swissadme.ch/index.php), cavernamines and cavernines were found to display a greater amount of water solubility compared to known monascus pigments; log P values are presented for selected pigments (Table 1). By virtue of its hydroxyl group, hydroxy-cavermanines display even lower log P than the other pigments.

TABLE 1
LogP values for selected A. cavernicola pigements
and corresponding Monascus pigments.

| | LogP (cal.) |
|---|---|
| Compound | (SwissADME) |
| trans-Cavernamine (FIG. 6) | 2.87 |
| Rubropunctamine from Monascus | 3.30 |
| trans-Cavernine (FIG. 9) | 2.91 |
| Rubropunctatin from Monascus | 3.29 |
| Hydroxy-cavernamine (FIG. 10) | 2.06 |

### Example 5. Coloration of Different Products Using Cis-Cavernamine-L

Cavernamine-L was prepared as described in example 1.8 and purified as described in example 1.4.

Colorimetric analysis was performed according to the CIEL*a*b*. CIE L*a*b* is the name of a color space specified by the International Commission of Illumination (CIE) and it includes all perceivable colors. The coordinate L* represents the lightness of the color (L*=0, yields black and L*=100 indicates diffuse white); and a* and b* represent the color-opponent dimensions: Red and green (a*) (negative indicate green, while positive indicate red), and yellow and blue (b*) (negative indicate blue and positive indicate yellow).

The system is based on the fact that light reflected from any colored surface can be visually matched by an additive mixture of the three primary colors: red, green, and blue. The L*a*b* model is a three-dimensional model, it can only be represented properly in a three-dimensional space.

CIELAB values were measured by Chroma Meter CR-200 by Konica Minolta. Measurements were done according to the manual. The perceptual color differences was calculated by taking the Euclidean distance ΔE* between the L*a*b* between two colors.

### 5.1 Coloration of Milk

Skim milk 1% from Arla was used to test the coloration with cis-cavernamine-L. Cavernamine-L powder was added in different concentrations to skim milk 1%. Milk and colored powder was mixed for 5 minutes before the solutions were subjected to colorimetric analysis according to the CIEL*a*b*.

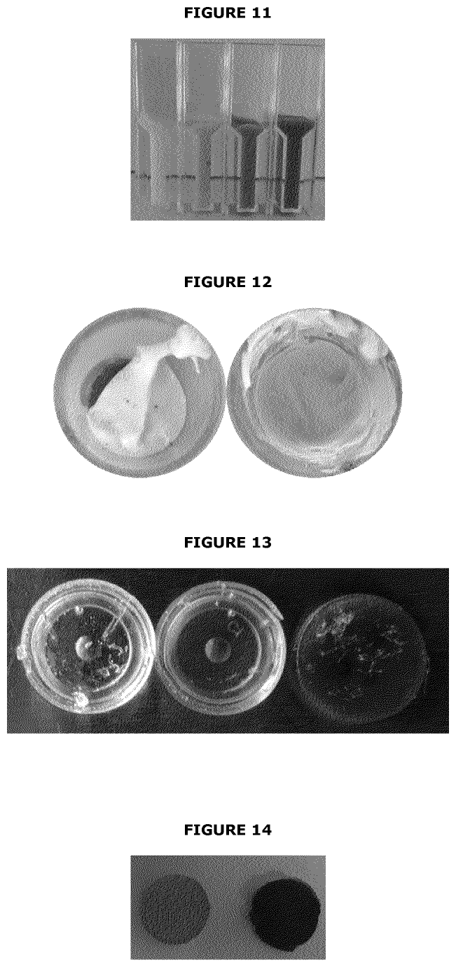

The coloration is visualized in FIG. 11, and the results of the colorimetric analysis are reported in Table 2.

TABLE 2
CIEL*a*b* color system measures of milk colored with
different concentrations of cis-cavernamine-L.

| Cavernamine-L | | |
|---|---|---|
| (PPM) | L*a*b* values | ΔE* |
| 0 | L: 86.02 | 0 |
| | a: −2.73 | |
| | b: −1.10 | |
| 28 | L: 78.36 | 12.07 |
| | a: 6.07 | |
| | b: 2.01 | |
| 140 | L: 53.89 | 37.37 |
| | a: 15.22 | |
| | b: 5.4 | |
| 280 | L: 52.04 | 40.74 |
| | a: 18.10 | |
| | b: 7.34 | |

### 5.2 Coloration of Skyr

Vanilla Skyr from Arla was used to test the coloration with cis-cavernamine-L.

Cavernamine-L powder was added to vanilla skyr from Arla. Skyr and colored powder was mixed for 5 minutes before the solutions were subjected to colorimetric analysis according to the CIEL*a*b*. The coloration is visualized in FIG. 12, and the results of the colorimetric analysis are reported in Table 3.

TABLE 3
CIEL*a*b* color system measures of skyr colored with
different concentrations of cis-cavernamine-L.

| Cavernamine-L | | |
|---|---|---|
| (PPM) | L*a*b* values | ΔE* |
| 0 | L: 95.39 | 0 |
| | a: −2.42 | |
| | b: 3.84 | |
| 46 | L: 86.89 | 12.48 |
| | a: 6.40 | |
| | b: 1.44 | |

### 5.3 Coloration of Epoxy

Two component epoxy resin system (PEBEO GEDEO 300 ml Cystal Resin), consisting of a resin and a hardener was bought from Pébéo.

Cavernamine-L powder was added to the hardner and mixed thoroughly. Colored and hardener were mixed 1:2 as per the use instructions, and allowed to harden for 24 hours. After it was hardend, the epoxy was subjected to colorimetric analysis according to the CIEL*a*b*.

The coloration is visualized in FIG. 13, and the results of the colorimetric analysis are reported in Table 4.

TABLE 4
CIEL*a*b* color system measures of epoxy colored with
different concentrations of cis-cavernamine-L.

| Cavernamine-L | | |
|---|---|---|
| (PPM) | L*a*b* values | ΔE* |
| 0 | L: 79.84 | 0 |
| | a: −0.79 | |
| | b: 0.47 | |
| 30 | L: 70.13 | 17.64 |
| | a: 4.81 | |
| | b: 14.09 | |
| 600 | L: 43.20 | 40.99 |
| | a: 15.33 | |
| | b: 9.32 | |

### 5.4 Coloration of Homemade Gummis

Gummi is a candy which is typically colored. In this example the ability of cavernamine-L ability to color homemade gummi was tested.

Gummi ingredient recipe: 14 g demineralized water, 7 g agar, 20 g sugar, 25 g glucose syrup, 1 g citric acid. Ingredients were mixed and heated to 65° C. for 30 minutes. Cavernamine-L powder was added to the mixture and stirred for 5 minutes at 65° C. The gummi mix was poured into mold and refrigerated for 24 h until they were firm. Gummies were subjected to colorimetric analysis according to the CIEL*a*b*.

The coloration is visualized in FIG. 14, and the results of the colorimetric analysis are reported in Table 5.

TABLE 5
CIEL*a*b* color system measures of gummies colored
with different concentrations of cis-cavernamine-L.

| Cavernamine-L | | |
|---|---|---|
| (PPM) | L*a*b* values | ΔE* |
| 0 | L: 41.66 | 0 |
| | a: 1.19 | |
| | b: 8.88 | |
| 180 | L: 31.74 | 22.96 |
| | a: 21.89 | |
| | b: 7.29 | |

### Example 6. Composition Comprising Cis-Cavernamine-L

Cavernamine-L was prepared as described in example 1.8 and purified as described in example 1.4.

Formulation of cavernamine-L with maltodextrin and citric acid. Pure cavernamine-L is too intense in its color to be practical to work with, as only miniscule amounts will need to be added to applications, making workflow harder. It is therefore ideal to dilute and formulate the color into a weaker intensity, such as illustrated below.

Dilution mixture was prepared as specified in table 6.

TABLE 6
Dilution mixture

| Ingredient | Amount | |
|---|---|---|
| Demineralized water | 1000 | g |
| Sodium citrate dehydrate | 16.9 | g |
| Citric acid | 8.1 | g |
| Maltodextrin | 25 | g |

The dilution mixture was adjusted to pH 5 with Sodium Hydroxide 2 M. The cavernamine-L powder was added to the dilution mixture in a concentration of 0.5 g/L and mixed for 5 minutes. The colored solution was then frozen prior to lyophilzation. Diluted red powder was recovered and the color intensity of the formulated cavernamine-L was detected to be E1% (at 492 nm) of 2.2, compared to E1% (at 492 nm) of 220 of original pure cavernamine-L powder.

1. A cavernamine pigment having the structure of Formula I or Formula II or a hydroxyl-derivative of said cavernamine pigment having the structure of Formula III: 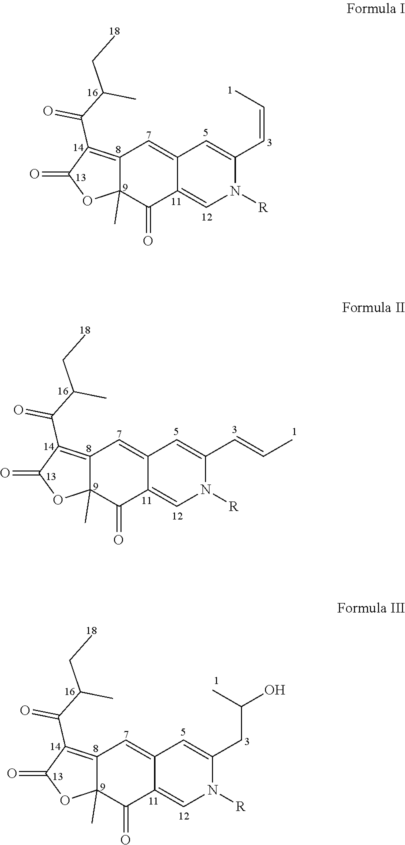
wherein R is hydrogen, or N—R is selected from among, an amino acid, a peptide, an amino sugar and a primary amine.

2. The cavernamine pigment having the structure of formula I or its hydroxyl-derivative having the structure of formula III according to claim 1, wherein N—R is an amino acid selected from the group consisting of: L-alanine, L-arginine, L-asparagine, L-aspartate, L-cysteine, L-glutamine, L-glutamate, L-glycine, L-histidine, L-isoleucine, L-leucine, L-lysine, L-methionine, L-phenylalanine, L-proline, L-serine, L-threonine, L-tryptophan, L-tyrosine, L-valine and L-ornithine.

3. An cavernine pigment having the structure of Formula IV or Formula V: 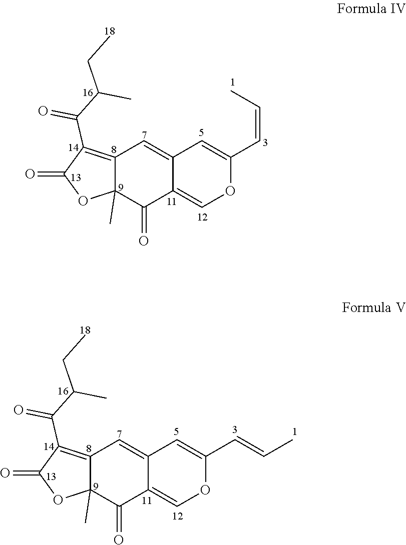 [SMILES: C/C=C\C1=CC2=CC3=C(C(=O)C(C)CC)C(=O)OC3(C)C(=O)C2=CO1 ; C/C=C/C1=CC2=CC3=C(C(=O)C(C)CC)C(=O)OC3(C)C(=O)C2=CO1]
wherein said cavernine pigment is a precursor of the cavernamine pigment and/or the hydroxyl-derivative of said cavernamine pigment according to claim 1.

4. A method for producing a cavernamine pigment and/or a hydroxyl-derivative of said cavernamine pigment according to claim 1 by fermentation, comprising the steps of:
a. providing spores or mycelia of a strain Aspergillus cavernicola,
b. cultivating said spores or mycelia in a liquid growth medium comprising a nitrogen source,
c. recovering the cavernamine pigment and/or its hydroxyl-derivative produced during said cultivating in step (b), and
d. optionally isolating said cavernamine pigment and/or its hydroxyl-derivative

5. The method for producing a cavernamine pigment and/or a hydroxyl-derivative of said cavernamine pigment by fermentation according to claim 4, wherein said cavernamine pigment has the structure of Formula I and its hydroxyl-derivative has the structure of formula III, and wherein N—R is selected from among an amino acid, a peptide, an amino sugar and a primary amine.

6. The method for producing a cavernamine pigment and/or a hydroxyl-derivative of said cavernamine by fermentation according to claim 5, wherein the sole nitrogen source in step (b) is a compound selected from the group consisting of a single amino acid, a peptide, an amino sugar and a primary amine.

7. The method for producing a cavernamine pigment and/or a hydroxyl-derivative of said cavernamine by fermentation according to any one of claims 4 to 6, comprising the additional step of:
a′) cultivating the spores or mycelia of step (a) in a preliminary liquid growth medium, wherein the sole nitrogen source of said preliminary liquid growth medium is an inorganic nitrogen source; and
wherein said step (a′) is followed by step (b).

8. The method for producing a cavernamine pigment and/or a hydroxyl-derivative of said cavernamine by fermentation according to claim 7, wherein the starting concentration of inorganic nitrogen in step (a′) is no more than 20 mM, continuing cultivation until the concentration of inorganic nitrogen is depleted to less than 5 mM.

9. The method according to any one of claims 4 to 8, wherein the liquid growth medium in step (b) is maintained within a pH of 4.0 to 6.5.

10. The use of a cavernamine pigment and/or a hydroxyl-derivative of said cavernamine according to claim 1 or 2 as a colouring agent for any one of a food, a non-food product and a cosmetic.

11. A composition comprising a cavernamine pigment and/or a hydroxyl-derivative of said cavernamine according to claim 1 or 2, wherein the composition is selected from among a food, a non-food product and a cosmetic.

12. A kit for coloring a composition, wherein the kit comprises (i) at least one cavernamine pigment and/or at least one hydroxyl-derivative of said cavernamine according to claim 1 or 2 and (ii) a stabilizing agent, wherein the pigment is supplied in a container, wherein the composition is selected from among a food, a non-food product and a cosmetic.

13. The use of a cavernine pigment according to claim 3 as a colouring agent for any one of a food, a non-food product and a cosmetic.

14. A composition comprising a cavernine pigment according to claim 3, wherein the composition is selected from among a food, a non-food product and a cosmetic.

15. A kit for coloring a composition, wherein the kit comprises (i) at least one cavernine pigment according to claim 3 and (ii) a stabilizing agent, wherein the pigment is supplied in a container, wherein the composition is selected from among a food, a non-food product and a cosmetic.
